(19) United States
(12) Patent Application Publication
(10) Pub. No.: US 2014/0012381 A1
(43) Pub. Date: Jan. 9, 2014

(54) JOINT IMPLANT AND A SURGICAL METHOD ASSOCIATED THEREWITH

(71) Applicant: OTB Surgical Designs Pty Ltd, Taringa (AU)

(72) Inventors: Robert David LABROM, Taringa (AU); Brett SPENCE, Auchenflower (AU); Declan Patrick BRAZIL, Cathswood (AU)

(21) Appl. No.: 13/934,743

(22) Filed: Jul. 3, 2013

(30) Foreign Application Priority Data
Jul. 5, 2012 (AU) 2012902895

(51) Int. Cl. A61F 2/44

(52) U.S. Cl. CPC A61F 2/446
USPC 623/17.16

(57) ABSTRACT

An implant able to be inserted into a surgically prepared joint space includes a body having a central cavity extending longitudinally through the body and at least one movable barb, and a cap for location within the central cavity at an end of the body. The location of the cap within the central cavity of the body causes outward movement of the barb.

### CROSS-REFERENCE TO RELATED APPLICATION

This application is related to, and claims priority in, Australian Patent Application No. 2012902895, filed on Jul. 5, 2012, the disclosure of which is incorporated in its entirety by reference herein.

### FIELD OF THE INVENTION

This invention relates to a joint implant and a surgical technique associated therewith. In particular the invention relates to spinal facet joint fusion and therefore will be described in this context. However, it should be appreciated that the implant may be used for fusing other joints throughout the body such as the radio-carpal joint, acromio-clavicular joint, carpal joints, metacarpal joints, tarsal joints, or any other synovial or fibrous joint in the skeleton.

### BACKGROUND OF THE INVENTION

Spinal fusion is a very common procedure performed via posterior surgical approaches for degenerative and deformity spinal pathologies. Spinal fusion can also address fusion of spinal levels adjacent to motion retaining devices/techniques. Spinal fusion limits motion between adjacent vertebrae to help eliminate pain arising from vertebrae applying pressure to a nerve root or neural element.

Typically posterior spinal fusion is achieved by inter-transverse process spinal fusion. This surgical technique often involves the placement of pedicle screws within vertebral bone and then attaching associated rods to associated pedicle screws. The pedicle screws in combination with the rods provide stability to the vertebrae so that bone graft can be placed between adjacent transverse processes and bone growth can occur to create permanent fusion of the spine.

Inter-transverse process spinal fusion is morbid with open approach surgical techniques. Accordingly, morbidity is reduced using more minimally invasive techniques to approach the posterior spinal elements. Further, bone graft delivery, containment, ectopic bone formation—especially with liquid bone morphogenic protein like substances, and resorption of loose bone graft remain problems with inter-transverse process spinal fusion.

Historically, posterior spinal fusions have also used a technique known as a Moe fusion (described by Dr John Moe). The surgical technique involves a partial destruction of the bony facet joint, decortication of surrounding bone surfaces, and insertion of non-structural bone chips/pieces into a space made after removal of the cartilage surfaces of the facet joint. There has even been the suggestion of surgical partial ablation of the joint with the use of an osteotome, gouge or bone nibbler.

This technique is not as frequently used today and the triple joint complex (i.e., the intervertebral disc space and the two facet joints) being fused may be biomechanically destabilised because of a space created between the facet joint surfaces, or worse, by the subtotal resection of the entire bony facet joint complexes. This technique leads to increased load sharing on any associated pedicle screw/rod construct and therefore may lead to increased loosening of such devices, and reduced fusion rates. However, there have been some advances in spinal facet fusions techniques.

US Patent Application No. 20060111782 and 20060111779 in the name of Petersen disclose minimally invasive spinal facet joint fusion. In particular, the patent applications disclose a facet joint fusion system that utilises a punch or drill that creates a hole through both sides of the spinal facet joint in a conical pattern. The hole is then filled with either the patients own harvested and compacted bone plug using iliac crest autograft, pre-made, pre-shaped cortical cadaveric allograft or pre-made, pre-shaped synthetic grafts.

The above technique works well in assisting in spinal facet joint fusion. However, the hole created in the spinal facet joint and filled by the bone plug may not be stable enough after surgery. The bone plug is relatively soft and therefore is able to be crushed with relative movement of the spinal facets. The minimisation of the hole created by compression of the bone plug may cause nerve compression which is undesirable. Pedicle screws and rods are therefore often required with this type of surgery and loosening of the screws in the pedicles in this setting would be undesirable and probable.

US Patent Application No. 20060085068 in the name of Barry discloses spinal facet joint implants and an associated method of non-invasive surgery to locate these implants within a spinal facet joint. The method includes the use of a guide wire to locate the implants in position within a spinal facet joint. Subsequently, each of the spinal facet joints has a hole that extends through the spinal facet joints. Hence, any application of a bone growth media to the implants to promote fusion has the potential to pass through the hole in the implant onto the underlying nerve root. This can cause damage to the nerve root which is undesirable.

US Patent application No. 20040111093 and 20060111782 in the name of Chappuis disclose a facet fusion system. In particular, the discloser relates to tapered implants placed within a surgically prepared spinal facet joint. The spinal facet joint system works reasonably well. However, the facet joint fusion time is relatively high as there are a limited number of fenestrations that extend through the implants that promote fusion. Further, many of the implants are solid which do not permit osteoinductive agents to be placed within the implants.

The reference to any prior art in this specification is not, and should not be taken as, an acknowledgement or any form of suggestion that the prior art forms part of the common general knowledge.

### OBJECT OF THE INVENTION

It is an object of the invention to overcome or alleviate one or more of the above disadvantages or provide the consumer with a useful or commercial choice.

### SUMMARY OF THE INVENTION

In yet another form, the invention resides in an implant able to be inserted into a surgically prepared joint space, the implant including:

a body having a central cavity extending longitudinally through the body and at least one large fenestrations extending laterally through the body, the central cavity and the at least one fenestration being communication with each other; and

a cap for location within the central cavity at an end of the body,

wherein the cap reduces the volume of the central cavity.

Preferably, the body and the cap is made from and/or coated with material that promotes bone growth such as hydroxyapatite, or a roughened external surface that promotes bone on-growth.

Normally the body of the implant is frusto conical in shape.

There is normally at least one barb that extends outwardly from a periphery of the body. There may be two, three or four barbs sets of peripheral barbs that extend. The barbs may be fixed or may be movable or a combination of fixed and movable barbs.

The central cavity normally is used to receive osteoconductive or osteoinductive agents. The body may have an end wall to hold the osteoconductive or osteoinductive agent within the implant.

Preferably, the body includes a skirt that extends around the body adjacent the distal end of the body.

Preferably, the body has at least two large fenestrations which are located on substantially opposite sides of the body. However, there may be three of four fenestrations that are equally spaced around the body.

The fenestrations may be sized to have an external surface area of at least 35% of the total external surface area of the implant. Preferably, the fenestrations may be sized to have an external surface area of at least 50% of the total external surface area of the implant. More preferably, the fenestrations may be sized to have an external surface area of at least 65% of the total external surface area of the implant, though a range of 35% to 70% will be likely.

Scraping slots may be located through the body adjacent the barb. The barb may be shaped to scape bone material. Subsequently, one or more of the barbs may have a scraping face. Preferably the barbs are shaped to scrape material into the central cavity when the implant is rotated.

Preferably, an external surface of body is tapered. The body may be of various shapes and could be trapezoidal, ovoid, cylindrical, or any other shape.

One or more channels may extend along an internal wall of the body.

The cap may be in the form of set screw. Accordingly, the cap may have an external thread. This external thread may engage with an internal thread located within the central cavity of the body. The cap may also include a rotation portion to enable the cap to be rotated. The rotation portion may be in the form of a rotation aperture. For example, the rotation aperture may be a hex lobe.

At least one movable barb may be located adjacent a top of the body. The cap being located within the central cavity may cause the movable barb to be displaced outwardly. Alternatively, the cap may prevent inward movement of the barb. There are normally at least two movable barbs and preferably at least four movable barbs that are effected by placing the cap within the central cavity of the body.

The implant may be constructed from materials including PEEK™ (oxy-1,4-phenyleneoxy-1,4-phenylene-carbonyl-1,4-phenylene), carbon fiber, metals such as titanium, stainless steel, chrome cobalt, and Nitinol, elastomer, silicone, bone cement, or plastics, TCP-tricalcium phosphate, BCP—bicalcium phosphate, HA—hydroxyapatite or combination of the above.

The implant may be made of a material and/or have design features that permit a degree of motion to occur through or around the implant such that it permits an environment suitable for dynamic fusion. Such an implant may be used in combination with dynamic posterior fusion constructs.

The implant may have any combination of holes or pores or gaps that permits bone to grow through the device and the easy passage of osteoinductive agents.

In another form, the invention resides in an implant able to be inserted into a surgically prepared joint space, the implant including:

a body having a central cavity extending longitudinally through the body and at least one movable barb; and

a cap for location within the central cavity at an end of the body,

wherein location of the cap within the central cavity of the body causes outward movement of the barb.

In one form, although not necessarily the only or broadest form, the invention resides in a method of performing surgery to enable joint fusion the steps including:

preparing bony surfaces of a joint to create an enlarged space between sides of the joint in which subchondral bone of the joint is exposed;

inserting a body of an implant, having at least two large fenestrations which are located on substantially opposite sides of the implant, into the enlarged space so that the implant contacts the subchondral bone;

orientating the implant so that the large fenestrations are located adjacent the subchondral bone on respective sides of the joint; and

locating a cap of the implant within a central cavity of body to displace a movable barb within the subchondral bone of the joint.

Preferably, once the implant is located within the joint, a hollow cavity of the implant is filled with an oesteoconductive agent so that the oesteoconductive agent contacts the subchondral bone surfaces through the large fenestrations. An osteoinductive agent may also be added to the implant and be contained within a sponge. The sponge may be compressed within the implant. The graft composite within the hollow implant may contain any osteoinductive material such as bone morphogenic protein, or similar.

The oesteoconductive agent may include bone graft material e.g. autograft, allograft, bone mineral substitute (TCP-tricalcium phosphate, BCP—bicalcium phosphate, HA—hydroxyapatite).

The surgical steps may be performed in an open or minimally invasive environment. The surgical steps may include utilising computerized and/or combined fluoroscopic navigation to assist in accurate placement of the trial or final implants.

The bony surfaces of the spinal facet joint may be manually and/or mechanically prepared. The preparation of the bony surfaces may include burring, drilling, taping, rasping, broaching and/or reaming.

Preferably, milling of the bony surfaces of the joint is performed to obtain a bone hole. The orientation of the bone hole may be made through a highly variable range of trajectories relative to the plane of an articular surface of the joint. The trajectory may be varied from parallel to the articular surface of the joint through to perpendicular to the articular surface of the joint.

The patient may be moved to a surgical position to distract the joint.

The implant may be inserted via a driving force. Alternatively, the implant may be inserted using a rotational force.

The implant may distract and fuse the joint.

In yet another form the invention resides in a method of performing surgery to enable joint fusion the steps including:

preparing bony surfaces of a joint to create an enlarged space between sides of the joint in which subchondral bone of the joint is exposed;

inserting a body of an implant, having at least two large fenestrations which are located on substantially opposite sides of the implant, into the enlarged space so that the implant contacts the subchondral bone;

orientating the implant so that the large fenestrations are located adjacent the subchondral bone on respective sides of the joint; and

locating an oesteoconductive agent within the central cavity of the body of the implant

locating a cap of the implant within a central cavity of body to reduced the volume of the central cavity to apply pressure to the oesteoconductive material.

### BRIEF DESCRIPTION OF THE DRAWINGS

Embodiments of the invention will be described with the reference to the accompany drawings in which:

FIG. 1A is a perspective view of a first embodiment facet joint implant;

FIG. 1B is further perspective view of a first embodiment facet joint implant;

FIG. 1C is a top view of the facet joint implant of FIG. 113,

FIG. 1D is a front view of the facet joint implant of FIG. 113,

FIG. 1E is a side sectional view of the facet joint implant of FIG. 1B,

FIG. 1F is detail view of the facet joint implant of FIG. 1F.

FIG. 2A is further perspective view of a first embodiment facet joint implant;

FIG. 2B is a top view of the facet joint implant of FIG. 2A;

FIG. 2C is a top view of the facet joint implant of FIG. 2B,

FIG. 2D is a side sectional view of the facet joint implant of FIG. 2A; and

FIG. 2E is detail view of the facet joint implant of FIG. 2F.

### DETAILED DESCRIPTION OF THE PREFERRED EMBODIMENT

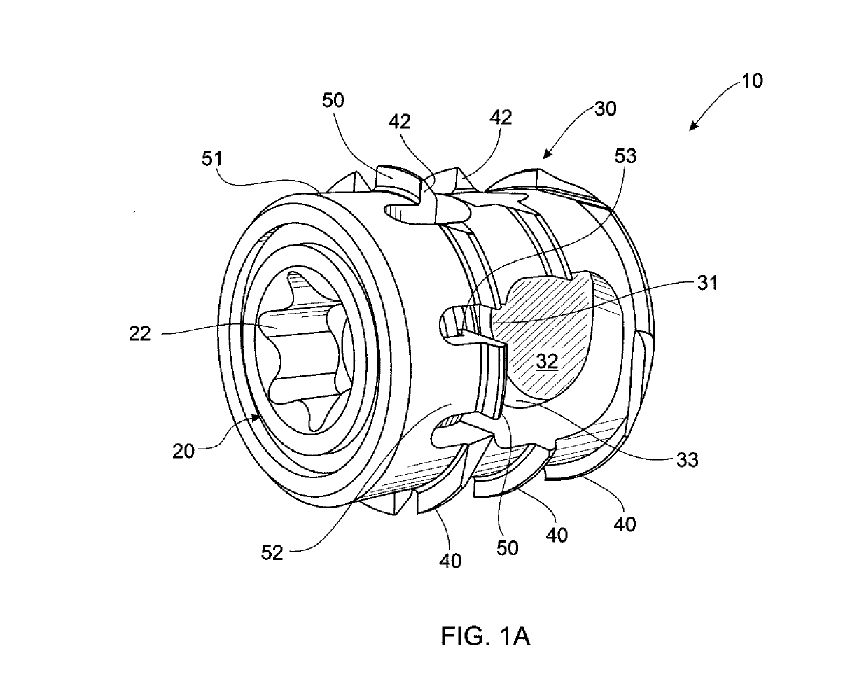

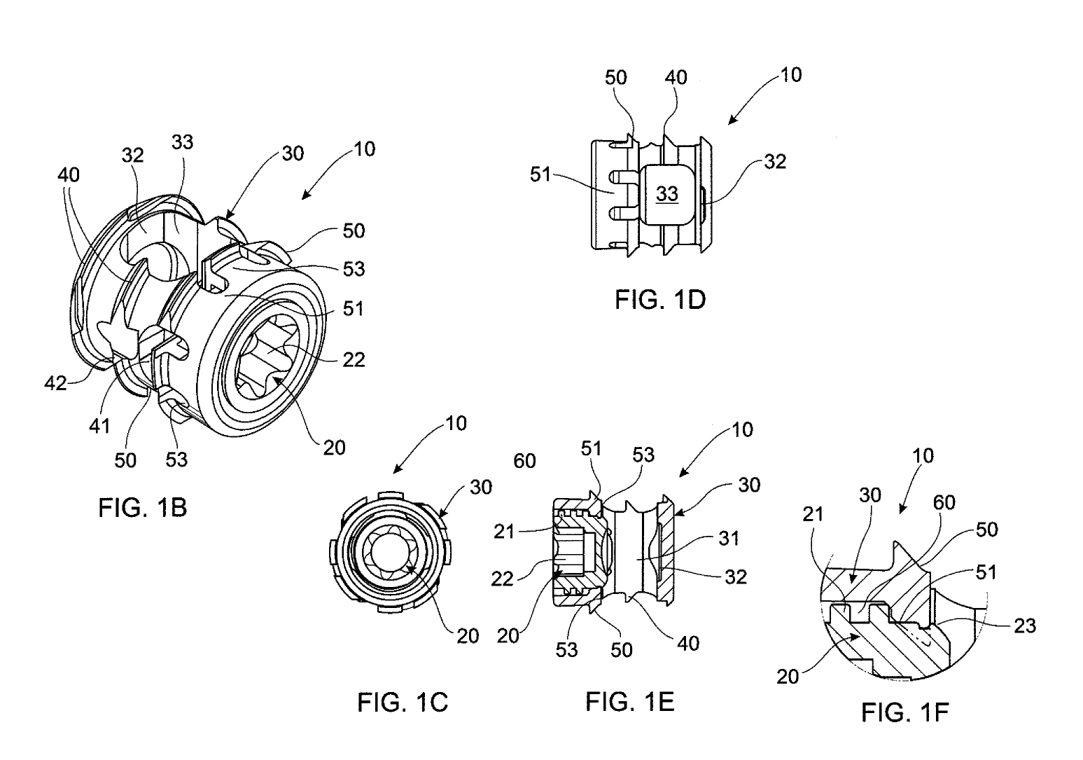

FIGS. 1A to 1F show an implant 10 able to be inserted into a surgically prepared spinal facet joint. It should be appreciated that even though this implant 10 has been specifically developed for use in surgically prepared spinal facet joints, it may have applications in other areas of the body such as the radio-carpal joint, acromio-clavicular joint, carpal joints, metacarpal joints, tarsal joints, or any other synovial or fibrous joint in the skeleton.

The implant 10 is made from titanium and may be coated with hydroxyapatite, or treated with a roughening technique such as acid/alkali treatments to promote a surface that enables bone on-growth. The implant 10 includes a cap 20 and a body 30.

The cap 20 is cylindrical in shape as is sized to fit within the body. An external thread 21 is located on a lower portion of the cap. A hex lobe 22 is located on an upper portion of the cap 20 to enable rotation of the cap 20. A retention groove 23 is located at the end of the external thread 21.

The body 30 is frusto conical in shape. That is, the body 30 is tapered from a top of the body 30 to a base of the body 30. A hollow central cavity 31 extends through a centre of the body 30. A top of the central cavity 31 is internally threaded. An end wall 32 is located adjacent the end of the body 30 at the bottom of the central cavity 31. Two large fenestrations 33 extend through the body 30 and are in communication with the central cavity 31. The large fenestrations 33 have an external surface area ratio of approximately 65% of the total external surface area of implant 10.

A series of fixed, circumferential, spaced-apart, barbs 40 are located along the length of the body 30. Each barb 40 has a scraping face 42 which is able to engage with the spinal facet joint. A series of scraping slots 41 are located adjacent some of the scraping faces 42 of some of the barbs 40. The fixed barbs 40 are shaped so that rotation of the fixed barb 40 will cause the scraping face 42 to engage or scrape the spinal facet joints upon rotation of the body 30. The body 30 also includes a number of movable barbs 50 which are formed at to the top 51 of the body 30. Each movable bard 51 is connected to the top 51 of the body 30 by a web 52 that extends between the top 51 of the body 30 and the movable bard 50. Each of the movable barbs 50 has a barb section 53 which extends into the central cavity 31.

An internal thread 60 is located within the top 51 of the central cavity 31 of the body 30. The internal thread is sized to mate with the external thread 21 of the cap 20.

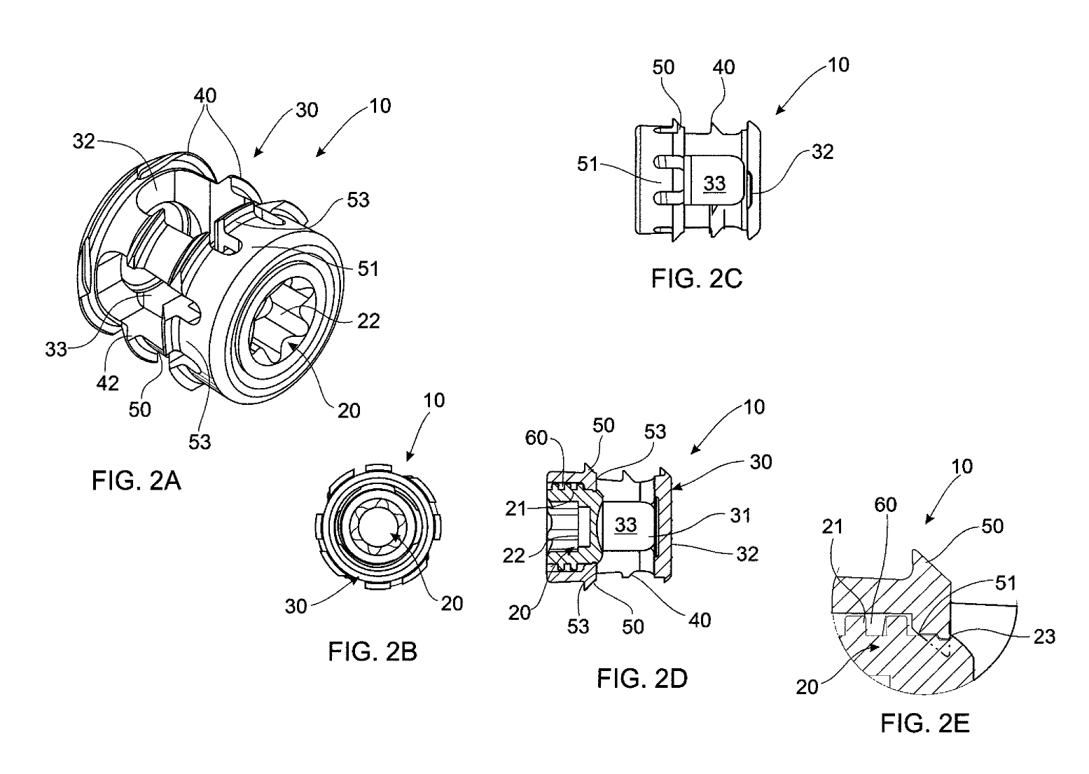

FIGS. 2A to 2E show an alternate embodiment of the implant in which there are four large fenestrations 33 instead of two large fenestrations 33.

In use, the implant 10 is typically used with spinal facet joint 20 that is required to be biologically fused in a surgical procedure. The facet joint 20 allows articulation between the vertebrae.

The surgical procedure commences by placing the patient prone or in a lateral position on an operating table. The skin and the deeper muscle layers of the patent are incised in a typical manner to partially expose the two spinal vertebrae so that access is provided to a facet joint0. It should be appreciated that minimally invasive surgical techniques may be utilised. The patient may be placed in a forward flexed lateral lying position to distract the spinal facet joints 20.

Further, the spinal facet joint 20 is further distracted using a distraction tool such as a double-action interspinous process manual distracter tool. Alternatively or additionally, an interspinous process spacer implant may be placed between the spinous processes of the inter-vertebral level to hold open the spinal facet joint.

It should be appreciated that moving the patient to a forward flexed lateral lying position and/or using a distraction tool and/or interspinous process spacer may not be necessary if the spinal facet joints are sufficiently distracted to provide access to the bony surfaces of the spinal facet joints.

Once the spinal facet joints are distracted somewhat, preparation of the bony surfaces of the spinal facet joints is commenced. Preparation involves burring, drilling, taping, rasping, broaching and/or reaming the bony surfaces of each spinal facet joint to create an enlarged spinal facet space. It should be appreciated that preparation of each spinal facet joint may be manually conducted or may use standard mechanical surgical tools such as a pneumatic drill or bone mill. Burring, drilling, taping, rasping, broaching and/or reaming is conducted on the bony surface of each facet joint until subchondral bone of each spinal facet joint is exposed.

It should be appreciated that preparation of the bony surface of each spinal facet joint is deliberate so that the enlarged spinal facet joint space is specifically shaped to receive the specifically shaped implant 10. For example, if the implant is frusto-conical in shape, a similarly frusto-conical enlarged milled joint shape space will be produced. Measuring tools to measure the size of the spinal facet joint space may be used such as a calliper and/or depth gauge to ensure the spinal facet joint space is correctly sized for the associated implant. A trial implant may be located within the spinal facet joint space to determine if the spinal facet joint space has been adequately prepared or alternately if a correctly sized implant has been chosen.

The ability to customize a spinal facet joint space with preparation of the bony surfaces to receive an implant 10 remains essential to the appropriate selection of an interposition facet joint implant 10 that may be either the same or a different size at each spinal facet joint pair level, depending upon that patients individual anatomy, size and possible spinal deformity.

Once the enlarged spinal facet joint space has been produced and measured, the implant 10 is then placed at the top of the surgically prepared spinal facet joints. An implant tool is then used to drive the implant 10 into the surgically prepared spinal facet joints. This can be achieved by either using hand force or using a mallet to hit the handle of the implant tool. As there is a series of circumferential barbs 40 and 50 that extend around the body 40 and 50, a stepped feeling is fed back through the tool as each barb enters the surgically prepared spinal facet joint.

Once the implant 10 is located within the surgically prepared spinal facet joints, the implant is rotated through between 45 to 90 degrees until the large fenestrations 33 are located on opposite sides of the joint. That is, the large fenestrations 33 are located adjacent the subchondral bone of the joint. The large fenestrations 33 provide the growth of new bone through the device, between each bony surface of the facet joint. That is, the large fenestrations 33 assists in fusion of the spinal facet joint.

The rotation of the implant also causes the scraping faces 42 of the barbs 40 to scrape bone material from the spinal facet joint that passes through the scraping slots 41 into the hollow cavity 31. The additional bone material through this auto-grafting technique also assists in fusion of the spinal facet joint. Further, rotation of the implant assists in preventing removal of the implant 10 from the spinal facet joints.

Additional oesteoconductive agent such as autograft, allograft, bone mineral substitute is located within the hollow cavity 31. The end wall 32 on the implant 10 prevents the oesteoconductive agent from falling through the central hollow cavity of the implant 10 onto the underlying nerve root. Once sufficient oesteoconductive agent is located within the central cavity 31, the cap 20 is screwed into the central cavity 31. That is, the external thread 21 of the cap 20 engaging with the internal thread of the central cavity 31. The cap 20, when screwed into the central cavity 31 of the body 30, contacts the sections 53 of the movable barbs 50 which extend into the central cavity 31. This causes the web 51 to pivot with respect to the top 51 of the body 30 moving the movable barbs 50 outwardly to forcibly contact the sounding bone surfaces. The retention groove 22 is assists in preventing unwanted removal of the cap 20 from the body 30.

The large fenestrations 33 located within the sides of the implant 10 allow direct contact of the oesteoconductive agent with the subchondral bone surfaces of the facet joint. Because bone growth is promoted when under compressive loads, the hollow central cavity 31 is packed with bone graft material which is compressed when the cap 20 is located on the body 30 the location of the cap 20 on the central cavity 31 reduces the volume of the central cavity 31. By compressing the bone graft material against the subchondral bone this ensures the best possible conditions for fusion to occur.

The frusto conical shape of the body 30 assists in maintaining contact between the two adjacent facet joints which is necessary to achieve good fusion. The fixed barbs 40 and movable barbs 50 assist in preventing unwanted removal and movement of the implant 10 which again essential for good fusion. The fenestrations 33 located within the implant allow bone growth through the body 30 yet again in order to achieve good fusion. The implant 10 is also structural in nature. That is, it cannot be substantially crushed and provides support to the spinal facet joint. Further, the implant provides distraction of the spinal facet joint.

Typically additional fixing devices such as the use of anterior interbody graft/cage/ramp fixation and/or posterior dynamic stabilization devices (pedicle screw based, or interspinous process based, or similar) are also utilised to at least temporarily or permanently stabilise the spinal facet joint to assist in fusion.

Further, any osteoinductive material and/or solution and associated carrier vehicles to augment the chances of a successful biological fusion is typically located adjacent the spinal facet joint. Such osteoinductive materials include BMP, OP1, bone marrow aspirate, and other autologous growth factors, including collagen sponges or similar delivery vehicles.

The procedure can combine the placement of posterolateral on-lay graft material between the transverse processes at the same spinal level to enhance fusion.

The procedure can combine the placement of interbody grafts or cages at the vertebral level being fused.

The spinal facet joints in the lumbar, thoracic, and cervical spine are relatively large surface areas of bone that normally load under compression in vivo, which is ideal for achieving bony fusion, with the use of implant once the cartilage and subchondral bone has been exposed. Removal of the cartilage surfaces and the subchondral bone leaves an enlarged spinal facet joint space that lends to an implant being inserted to share load in compression which is a normal biomechanical feature in standing, walking and even lying down.

The above spinal fusion surgery can be performed via minimally invasive surgery techniques that can reduce morbidity, save on patient hospital stays, and reduce associated complications.

The facet joint in the lumbar spine is on average 16 mm long and 14 mm wide and has an average surface area of 160 mm², assuming an ovoid shape. Retention of the bony co-planar spinal facet joint surfaces, or a specifically reciprocally milled shape, adds to biomechanical stability of the triple joint and load sharing between any additional implants. Further, bleeding bone surfaces under compression, with a suitable implant with large fenestrations is likely to have a high fusion rate.

A distractive force may be applied to the facet articular processes either by patient positioning in a forward flexed posture, distraction through the pedicle screw and rod construct, or via a distractive force between the spinous processes at the level(s) being fused. Such a spinal facet joint interposition implant technique can exist without additional distraction of the spinal facet joint.

Each patient has slightly different anatomical features with regards their spinal facet joints with regards size and shape, and there may even be variation between two facet joints at the one spinal level. Surgical customization of the prepared bone surfaces between two facet joint articular processes can enable the appropriate selection of an interposition facet joint implant.

The solid nature of the interposition facet implant adds to the load sharing between it and any pedicle screw construct posteriorly, or cage/graft anteriorly between the two vertebral bodies being fused.

Pre-operative planning of the facet joint is easily obtained with routine radiological investigations (CT, MRI) and hence allows an indication of the size of the graft/implant/device needed.

The spinal facet joint can be easily assessed for degrees of biological fusion after insertion of an interposition facet implant using radiology techniques such as CT, MRI, and X-ray.

By having a known size of interposition facet implant, the surgeon will now have the ability to compare surgical techniques between patients and therefore permit more generalizable techniques that can be more easily scientifically compared.

In this specification, the terms “comprise”, “comprises”, “comprising” or similar terms are intended to mean a non-exclusive inclusion, such that a system, method or apparatus that comprises a list of elements does not include those elements solely, but may well include other elements not listed.

It should be appreciated that various other changes and/or modifications may be made to the embodiments described without departing from the spirit or scope of the invention.

1. An implant able to be inserted into a surgically prepared joint space, the implant including:
a body having a central cavity extending longitudinally through the body and at least one movable barb; and
a cap for location within the central cavity at an end of the body,
wherein location of the cap within the central cavity of the body causes outward movement of the barb.

2. The implant of claim 1 wherein at least one large fenestration extends laterally through the body, the at least one large fenestration being in communication with the central cavity.

3. The implant of claim 2 wherein there are two fenestrations that extend laterally through the body, the two fenestration being in communication with the central cavity.

4. The implant of claim 3 wherein the two large fenestrations are located on substantially opposite sides of the body.

5. The implant of claim 1 wherein the body is frusto conical in shape.

6. The implant of claim 1 wherein the at least two movable barbs, each of the barbs being movable outwardly with the location of the cap within the central cavity of the body.

7. The implant of claim 1 wherein there at least four movable barbs, each of the barbs being movable outwardly with the location of the cap within the central cavity of the body.

8. The implant of claim 1 including at least one fixed barb that extends outwardly from a periphery of the body.

9. The implant of claim 1 wherein the body includes a skirt that extends around the body adjacent the distal end of the body.

10. The implant of claim 1 wherein the cap is in the form of set screw.

11. The implant of claim 1 wherein the cap has an external thread which engages with an internal thread located within the central cavity of the body.

12. The implant of claim 1 wherein the cap includes a rotation portion to enable the cap to be rotated.

13. The implant of claim 12 wherein the rotation portion is in the form of a rotation aperture.

14. The implant of claim 1 wherein the at least one movable barb is located adjacent a top of the body.

15. An implant able to be inserted into a surgically prepared joint space, the implant including:
a body having a central cavity extending longitudinally through the body and at least one large fenestration extending laterally through the body, the central cavity and the at least one fenestration being in communication with each other; and
a cap for location within the central cavity at an end of the body,
wherein the cap reduces the volume of the central cavity.

16. A method of performing surgery to enable joint fusion the steps including:
preparing bony surfaces of a joint to create an enlarged space between sides of the joint in which subchondral bone of the joint is exposed;
inserting a body of an implant, having at least two large fenestrations which are located on substantially opposite sides of the implant, into the enlarged space so that the implant contacts the subchondral bone;
orientating the implant so that the large fenestrations are located adjacent the subchondral bone on respective sides of the joint; and
locating a cap of the implant within a central cavity of body to displace a movable barb within the subchondral bone of the joint.

17. The method of claim 16 including the step of:
filling the central cavity of the implant with an oesteoconductive agent so that the oesteoconductive agent contacts the subchondral bone surfaces through the large fenestrations.

18. The method of claim 16 including the step of adding an osteoinductive agent to the central cavity of the implant, wherein the osteoinductive agent is contained within a sponge.

19. The method of claim 18 wherein the sponge is compressed by the cap.

20. A method of performing surgery to enable joint fusion the steps including:
preparing bony surfaces of a joint to create an enlarged space between sides of the joint in which subchondral bone of the joint is exposed;
inserting a body of an implant, having at least two large fenestrations which are located on substantially opposite sides of the implant, into the enlarged space so that the implant contacts the subchondral bone;
orientating the implant so that the large fenestrations are located adjacent the subchondral bone on respective sides of the joint; and
locating an oesteoconductive agent within the central cavity of the body of the implant
locating a cap of the implant within a central cavity of body to reduce the volume of the central cavity to apply pressure to the oesteoconductive material.
